(19) United States
(12) Patent Application Publication
(10) Pub. No.: US 2026/0320344 A1
(43) Pub. Date: Oct. 15, 2026

(54) SYSTEM FOR DIRECT NEURAL INTERACTION BETWEEN INDIVIDUALS USING REAL-TIME SIGNAL EXTRACTION, TRANSMISSION, AND NEURAL SYNCHRONIZATION - BRAIN-TO-BRAIN COMMUNICATION TECHNOLOGY

(71) Applicant: Juliana Lourenco Lopes Demasi, San Francisco, CA (US)

(72) Inventor: Juliana Lourenco Lopes Demasi, San Francisco, CA (US)

(21) Appl. No.: 19/466,127

(22) Filed: Jan. 30, 2026

Related U.S. Application Data
(60) Provisional application No. 63/752,756, filed on Feb. 2, 2025.

(51) Int. Cl. G06N 3/00; H04L 9/40

(52) U.S. Cl. CPC G06N 3/002; H04L 63/0428

(57) ABSTRACT

The present invention refers to a brain-to-brain communication technology, and relates to neurotechnology, neuroscience, hyperpolarized MRI, artificial intelligence (AI), functional magnetic resonance imaging (fMRI), communication and psychology, for direct brain-to-brain (B2B) communication. The system comprises: a real-time Functional MRI (fMRI) processing via Pyneal Toolkit or proprietary software; a hyperpolarized Carbon 13 MRI (HP-13C MRI) module for tracking cognitive metabolic activity; an AI-powered Neural Processing Unit (NPU) to optimize neural transmission; and a secure cloud-based neural data transmission network, wherein the receiver's brain processes the incoming signal without hardware external stimulators.

### CROSS-REFERENCE TO RELATED APPLICATIONS

This application claims the benefit under 35 U.S.C. § 119(e) of U.S. Provisional Application Ser. No. 63/752,756, filed Feb. 2, 2025, the contents of which is hereby incorporated by reference in its entirety.

### FIELD OF APPLICATION

The present invention relates to the field of neurotechnology, neuroscience, medicine, psychology, hyperpolarized MRI, artificial intelligence (AI), functional magnetic resonance imaging (fMRI), for direct brain-to-brain (B2B) communication.

### BACKGROUND OF THE INVENTION

Traditional communication methods rely on verbal, visual, or written language to transmit information between individuals. Brain-to-brain communication (B2B) has been theorized as a potential form of direct neural interaction, but current methods face challenges such as latency, signal noise, and external stimulation dependencies.

Current research in brain-to-brain (B2B) communication and brain-computer interfaces (BCIs) faces several key limitations:
1. latency issues—real-time fMRI signal processing faces delays, limiting practical applications;
2. dependence on external stimulators—existing systems require external electrical/magnetic stimulation (TMS, EEG, BCIs);
3. lack of metabolic validation—many systems fail to confirm whether the receiver's brain actually interprets the signal, and
4. limited scalability for long-distance communication—most current B2B systems operate only in short-range laboratory environments.

The technology of the present invention enables non-verbal, long-distance neural synchronization between individuals using AI-driven real-time fMRI processing, metabolic imaging (Carbon 13 MRI or HP-13C MRI), and the Pyneal Toolkit or proprietary software for real-time fMRI analysis. This technology represents a new and emerging solution that has never been fully realized before. In the context of the present invention, the Carbon-13 MRI can be used to verify brain metabolic alignment between sender and receiver. It creates biological feedback for the AI yp adapt and optimize neural transmissions.

While there have been early-stage experiments in brain-to-brain communication, the specific combination of hyperpolarized MRI (HP-13C MRI), metabolic tracking, and real-time cloud transmission (DICOM) is unprecedented.

Specifically, it provides a system for direct neural interaction between individuals using real-time signal extraction, transmission, and neural synchronization.

This new technology is called MINDNET. The sender and the receiver, when using the technology (MINDNET) are considered synched in through the integration of Pyneal Toolkit or proprietary software, fMRI, Hyperpolarized MRI (HP-13C MRI) and AI-enhanced neural synchronization, where the receiver's brain processes the incoming signal without hardware external stimulators.

The Sender: a human Radio Station: imagine a person's brain is like a radio station: the brain emits specific signals (neural patterns and metabolic rhythms). These signals represent useful information like a music being broadcast that can be listened by a Hyperpolarized MRI Technology resource center, as an example.

The system consists of the following core components: a Sender's Brain Signal Processing through fMRI (AI), a Neural Transmission & Signal Encoding (AI) through fMRI and a Receiver's Brain as a Natural Decryption System.

With the Sender's Brain Signal Processing (AI) the sender is synched in the Functional MRI (fMRI)+HP-13C MRI system that scans the sender's neural and metabolic activity. Additionally, the Pyneal toolkit or proprietary software extracts real-time BOLD (blood-oxygen-level-dependent) and metabolic patterns, and an AI-based encoding system converts the neural signals (message) into a format optimized for transmission.

In the MindNet system, the “radio signal” comprises complex neural, metabolic—and potentially ultra-weak microwave electromagnetic-information from the sender's brain. The AI's noise reduction function is critical: it isolates the therapeutic signature (e.g., low pain/low inflammation neural state) from irrelevant data (scanner artifacts, physiological rhythms, ambient EM, iatrogenic signals, weak microwave emissions). Drawing on the Bryukhovetskiy et al. (2020) finding of microwave brain emissions, we recognise that the dimensionality of the signal may be even higher than typical neuroimaging, further underscoring the need for advanced filtering, feature extraction, classification, and metabolic confirmation. Only through such rigorous noise reduction can the receiver's brain tune into a clear, meaningful broadcast capable of inducing the desired therapeutic state.

The Neural Transmission & Signal Encoding makes a cloud-based neural network securely transmits the encoded neural signals to the receiver, and the signal is encrypted using a brain-adaptive encoding method, ensuring only the receiver's nervous system can process and interpret it.

With the Receiver's Brain as a Natural Decryption System, the receiver is synched in the fMRI+HP-13C MRI system that detects incoming neural patterns. Instead of using external devices to decode the signal, the receiver's brain naturally interprets the message based on shared neural representations, and AI models assist in optimizing alignment and adjusting signal strength, improving natural interpretation.

MindNet relies on measured signals (fMRI, HP-13C MRI, EEG):
The system uses AI+cloud encryption to transfer brain states, like a secure radio transmission, not ambient field detection;
The platform incorporates feedback and metabolic alignment, which is measurable and verifiable with current medical tech.

To ensure effective brain-state transfer, the AI system must isolate only the relevant neural and metabolic information. This process is what we define as noise reduction—and it is not just a feature, but the foundational function that allows MindNet to operate safely and precisely.

Much like a radio tuner cannot decode music from a static-filled frequency, MindNet cannot effectively induce therapeutic brain alignment unless the input signal is pure. Any misread patterns caused by unfiltered noise could:
cause ineffective or even contradictory brain alignment,
mislead clinical interpretation, and
reduce safety, reliability, and trust in the system

Thus, signal purification isn't optional—it's what makes MindNet's brain-to-brain communication scientifically grounded and clinically viable.

“Noise reduction” in MindNet is the AI's intelligent filtration of complex brain data to isolate only the biologically meaningful, replicable state needed for therapeutic transmission. It ensures:
precision in signal transmission,
safety in cross-brain alignment,
integrity in neural pattern replication, and
scalability of the system across subjects and contexts

Without it, the signal is too impure to be useful—with it, MindNet becomes a true brain-state delivery platform.

MindNet's AI performs a critical filtering operation to separate signal from noise:
it applies dimensionality reduction (e.g., PCA, ICA) to disentangle overlapping sources;
it uses supervised learning to classify signal patterns based on desired outcomes (e.g., low pain, calm focus, etc.);
it leverages temporal smoothing and frequency filtering to exclude transient disruptions; and
it verifies results through cross-modality confirmation (e.g., comparing HP-13C metabolic profiles to BOLD dynamics).

Through this multi-layered process, the AI doesn't merely “read” the brain—it understands which parts of the signal matter, and which are biologically irrelevant.

In MindNet, the signal to be transmitted isn't only the BOLD and metabolic signature captured via fMRI/HP-13C MRI—it may also include ultra-weak brain-emitted microwave EM components as suggested in the Bryukhovetskiy et al. study. This increases the necessity for sophisticated noise reduction, because the relevant brain-state signature is buried among far stronger non-therapeutic signals, ambient EM noise, scanner artifacts, physiological rhythms, and metabolic variation. The AI must thus perform dimensionality reduction, artifact rejection, spectral filtering (including microwave band), supervised classification, and metabolic validation, ensuring that only the pure, therapeutic “broadcast” is sent.

The core noise-reduction argument remains centered on well-accepted modalities (fMRI BOLD, metabolic imaging, neural entrainment), but can mention the microwave emission study as an emerging layer of complexity.

### Noise Reduction in MindNet: Safeguarding Signal Integrity Across Modalities

In the MindNet system, “noise reduction” is the AI-driven process that isolates biologically meaningful brain-state information from irrelevant or disruptive signal components. This function is not simply technical optimization—it is the core mechanism that ensures precision, safety, and therapeutic accuracy when transmitting a brain's state from one individual to another.

Whether working with functional MRI (fMRI), hyperpolarized Carbon-13 MRI, or electromagnetic signatures, the human brain generates a dense, multimodal signal field. Most of this information is not therapeutically useful, and some may even interfere with correct interpretation. Noise reduction allows MindNet to extract the essence of a healthy neural pattern—and discard everything else.

### Sources of Noise in Brain Signal Transmission

The brain's activity is captured using advanced imaging techniques like:
fMRI (BOLD signal): Sensitive to blood oxygenation, but also prone to artifacts from motion, respiration, and scanner instability.
HP-13C MRI: Measures real-time metabolism but can be contaminated by transient chemical shifts.
Electromagnetic emission recordings: As described by Bryukhovetskiy et al. (2020), the human brain may also emit ultra-weak microwave radiation in the 1.5-4.5 GHz range, potentially reflecting cognitive processes.

Each of these domains is rich in data but also cluttered with physiological, environmental, and machine-related noise. Without filtering, this complexity makes the signal unreadable and unusable.

This process is comparable to an intelligent radio tuner that knows how to ignore static, filter interference, and lock on to the exact transmission needed—only here, it's doing so across three or more signal types simultaneously.

Without rigorous noise reduction, MindNet would be attempting to transmit brain data through static. With it, the system becomes a precision instrument for brain-state replication. Without it, the system would be ineffective. With it, MindNet becomes a safe, scalable, and science-aligned method of brain-to-brain communication and pain-state correction.

Unlike traditional brain-computer interfaces (BCIs) that require external decoding hardware, this system leverages the nervous system's inherent ability to process and interpret neural patterns through Mirror Neuron Activation; Neural Entrainment & Synchronization and Metabolic Signal Confirmation (HP-13C MRI).

The fMRI signal—especially when augmented with hyperpolarized \hat{ }13C MRI-captures real-time metabolic and neural activity with high fidelity. This is the radio tower of the MindNet metaphor. In this analogy:
the “radio wave” is the multi-dimensional brain data captured from a sender: including neural activation (fMRI) and metabolic status (HP-13C);
the broadcast comes from a powerful Hyperpolarized MRI Center, a world leader in carbon-13 metabolic imaging like the one in San Francisco, USA;
this signal contains the “brain state” of the sender, including potentially therapeutic states (like pain-free, calm, focused, etc.), and
it is encoded and cleaned up by AI-driven noise reduction so that only the essential information is preserved—like a clear radio frequency amid static.

The Mirror Neuron Activation makes mirror neurons replicate and interpret brain activity from another person; the receiver's neurons fire in response to the sender's data, allowing perception of the transmitted information. For example: the sender thinks about a moving object, and the receiver's visual cortex activates, as if seeing it themselves.

### How the User (Receiver) is “Synched In” to the Signal

1. To receive the “broadcast,” the user must also be connected to Mindnet—a fMRI system capable of decoding signals in real time. This means:
The receiver brain is fed with the signal (visually, auditorily, or directly into interface layers like neurofeedback loops).
Optionally, a closed-loop system may run comparisons between sender & receiver signals to gradually “tune” the receiver's brain state to match the sender's (biofeedback via AI loop).

2. Just as a radio uses internal circuitry to “tune” to a specific station, MindNet uses:
AI signal processing to align the relevant neural or metabolic features;
Deep-learning algorithms trained on large fMRI datasets to interpret, translate, and re-project the sender's brain state into meaningful instructions or stimulus for the receiver;
This tuning could lead to entrainment, neural mirroring, or brain-to-brain stimulation effects where the user's brain naturally resonates with the incoming data.

3. Therapeutic Signal Alignment:

If the sender's brain state is “ideal” (low inflammation, pain-free, clear cognition), then the receiver's brain—by syncing with that state—could:
Down-regulate pain and even inflammation, following known CNS-metabolism pathways;
Mimic therapeutic patterns, reinforcing the desired state via Hebbian plasticity (“neurons that fire together, wire together”);
Be part of a new paradigm in neuro-therapy where a patient receives optimal “brain states” as medicine.

With the Neural Entrainment & Synchronization [System] the receiver's brainwaves naturally align with the incoming signals and an AI-enhanced Pyneal or proprietary software analysis ensures real-time synchronization, improving accuracy.

With the Metabolic Signal Confirmation, the HP-13C MRI tracks real-time metabolic activity in the receiver's brain, and a real cognitive response confirms accurate interpretation of the incoming signal. This ensures that the receiver is truly understanding the data of the sender, rather than random neural noise.

To enhance natural interpretation and neural synchronization, the system integrates AI-driven optimization models within Pyneal Adaptive Neural Encoding or proprietary software, which makes the AI to dynamically adjust the transmission signal to match the receiver's cognitive rhythm; a predictive software learning improves alignment over repeated transmissions. Additionally, there is a Real-Time Feedback for Signal Clarity, as the AI tracks metabolic activation in the receiver's brain adjusting signal strength accordingly, noting that unsuccessful transmissions trigger adaptive AI corrections, improving future accuracy.

To secure neural transmission & brain-based decryption, there are no external decryption devices; unlike traditional BCI systems that require computers to process signals, this approach allows (i) a direct neural signal interpretation by the receiver's nervous system and (ii) an end-to-end encryption using biological authentication (brain-only processing).

Therefore, with the system presented in this invention provides the results as follows:
sender's brain data are processed and interpreted naturally by the receiver's brain;
receiver's metabolic activity confirms true neural interpretation;
AI optimizes long-term neural synchronization for improved efficiency, and
there is no need for external devices to decode the signal-decryption is fully brain-based.

### 1. High-Resolution Brain Signal as a Transmissible Source

At its core, MindNet captures a powerful “signature” of a brain (Brain data) state using technologies like:
Functional MRI (fMRI): captures neural activation maps (oxygen use in different brain regions);
Hyperpolarized \hat{ }13C MRI: captures real-time metabolic activity (like glucose-to-lactate conversion), which reflects inflammation or tissue health.

Once these signals are acquired:
They form a multi-dimensional data structure that encodes brain data—that could be interpreted as biological well-being itself (pain-free and even anti-inflammatory states).
This is the neural “signal” MindNet intends to transmit.

Think of it like recording not just a melody, but the biochemical harmony of the brain while that melody is being played.

### 2. Transmitting the Signal: The Brain-as-Network Model

Here's how the signal becomes universally accessible:
a) Cloud-based Broadcasting through fMRI infrastructure:
   Just as GPS signals or Wi-Fi frequencies are distributed globally, MindNet transmits brain state data through encrypted cloud platforms.
   Medical-grade protocols like DICOM (used for MRI/PET data) ensure precise transfer of high-dimensional data.
   Advanced compression+encryption makes this secure, lightweight, and scalable-similar to telehealth imaging workflows, but live and dynamic.
b) Receiver end: signal integration:
   On the receiver side, even without a physical decoder in real time, the pattern can be:
   Reconstructed as neurofeedback stimulus (via EEG, TMS, VR headset, audio, etc.);
   Applied as digital therapeutic where AI reconstructs the brain state signal and presents it to the receiver's brain in modulated formats;
   The signal doesn't need to be decoded directly in the same way it was encoded—just like a .mp3 doesn't require the same instruments to recreate the music, AI can reformat brain states into entrainable outputs.
      3. How the Receiver's Brain “Syncs In” from Anywhere

Once the broadcast is received, AI reconstructs and simplifies the signal for delivery through different modalities-like:
Visual entrainment,
Auditory stimulation,
Brain-computer interfaces.

These delivery mechanisms guide the receiver's brain toward alignment, like tuning a musical instrument to match a frequency.

Entrainment and plasticity are key: when exposed to patterns of calm, pain-free brain activity, the receiver's brain begins to adopt similar neural rhythms and reduce pain, inflammation, or stress in a non-invasive way.

### 4. Why it Works: The Science of Brain Entrainment and Mirror Neurons

MindNet relies on well-established neuroscience principles:
Mirror neurons: Humans naturally mimic and internalize observed brain/emotional states.
Hebbian plasticity: “Neurons that fire together wire together”—the receiver's brain adapts over time to the signal.
Neurofeedback: Studies show that real-time training using remote brain signals can improve cognitive function, reduce chronic pain, and even modulate mood.

These allow the receiver's brain to be “trained” remotely—even if they are in another city or country—just like how remote meditation apps or cognitive training systems work, but using biologically grounded brain data instead of generic content.

MindNet makes pain relief and cognitive healing a globally deliverable signal, much like streaming a radio station. This allows:
Decentralized healing;
Scalable neurotherapeutics;
Global access to personalized neural medicine.

It's a shift from “healthcare in a hospital” to “healthcare by frequency.”

### How the Brain “Reads” the Signal

To understand how the AI signal is sent back to the brain and “read” by it, we need to dive into neuroscience, signal processing, and neuroplasticity. Here's a full explanation of how this feedback loop works in the MindNet system.

The brain doesn't literally decode digital data like a computer—but it responds to:
Patterns;
Repetition;
Synchronization.

This process is based on:

a. Hebbian Learning: “Neurons that fire together, wire together”.

If the incoming signal (e.g., alpha waves or metabolic cues) is present consistently while the user experiences a therapeutic state, the brain forms new connections.

b. Mirror Neuron System: The brain automatically mimics and internalizes observed patterns—if the AI presents brain-state patterns similar to those seen in empathy, safety, or relief, the receiver brain mirrors them.

The brain automatically mimics and internalizes observed patterns—if the AI presents brain-state patterns similar to those seen in empathy, safety, or relief, the receiver brain mirrors them.

c. Neuroplasticity+Entrainment: Entrainment is like syncing to a rhythm-when external stimuli follow a certain frequency (e.g., 10 Hz alpha), the brain starts producing similar internal rhythms.

Entrainment is like syncing to a rhythm—when external stimuli follow a certain frequency (e.g., 10 Hz alpha), the brain starts producing similar internal rhythms.

This is how calm, clarity, or pain-free states can be induced and learned.

The brain “reads” the AI-returned signal by entraining to it, mimicking it, and reconstructing its meaning through sensory, cognitive, and emotional responses-thanks to neuroplasticity, mirror neurons, and feedback training.

This is what turns MindNet into a non-invasive, cloud-powered, globally-accessible therapy system—powered by AI, validated by neuroscience, and scalable through neurofeedback technologies.

### What is Hebbian Learning?

Hebbian learning is a fundamental neuroscience principle summarized as: “Neurons that fire together, wire together.” (Donald Hebb, 1949).

It means that when two neurons activate simultaneously, the connection (synapse) between them becomes stronger. Over time, this leads to lasting structural and functional changes in the brain.

This is how learning, memory, and adaptation happen in the brain-even without conscious effort.

### The Brain's Own Electrical Activity

Crucially, the brain is already generating electrical signals at all times:
Brain cells (neurons) communicate using electrical impulses (called “action potentials”);
These signals create oscillating wave patterns (alpha, beta, gamma, delta) that can be measured with EEG or indirectly via fMRI;
These waves are not random—they reflect attention, mood, memory, and even physical pain.

So: the brain is an electrically active organ, constantly “broadcasting” its state internally and externally.

1. How Hebbian Learning Connects to this

MindNet leverages Hebbian learning by feeding the brain new, healthy patterns (from another brain's fMRI/metabolic signal) and helping the receiver brain “practice” those same patterns.

Here's what happens:
1. AI delivers a therapeutic brain pattern (e.g., from UCSF's fMRI signal of pain relief);
2. The receiver's brain is exposed to this pattern repeatedly via light, sound, or brainwave-based stimulation;
3. As the brain attempts to mimic or align with the pattern, its own neurons fire in sync with the therapeutic rhythm;
4. These new connections are reinforced by Hebbian plasticity;
5. Overtime, the receiver's brain rewires itself to reflect the new, healthier state.

This is neural learning without language-purely electrical and patterned.

2. Why this Matters for MindNet

Because the brain is already an electrochemical antenna, it doesn't need a chip or implant to “receive” a signal. It simply needs:
Repeated exposure;
Accurate pattern delivery;
AI feedback to maintain alignment.

Then Hebbian learning takes over-building long-term changes from short-term entrainment.

### 3. Final Insight

MindNet doesn't force the brain—it teaches it.

By gently synchronizing and reinforcing healthy patterns, MindNet activates the brain's own natural learning laws, using Hebbian plasticity+electrical synchrony to induce healing, pain relief, or emotional balance—all through signals alone, like tuning in a radio.

### Hebbian Learning: The Brain's Natural “Sync Engine”

Hebbian learning is based on the idea that neurons that fire together, wire together. When two regions of the brain are activated simultaneously (or in a specific pattern repeatedly), they:
Strengthen their connection
Form long-term memory and behavioral patterns;
“Learn” new states like calm, pain reduction, or enhanced function.

This is how habits, motor skills, and even emotional responses are formed.

In the MindNet model, a Hyperpolarized fMRI platform becomes the equivalent of a powerful radio transmitter, generating therapeutic “brain state signals.”

### 4. Why it Works Globally

Because the brain is already an electrochemical antenna, it can:
Detect incoming patterns (via eyes, ears, skin, or cortical stimulation);
Respond by firing neurons in synchrony;
Strengthen those circuits through Hebbian learning.

This means that Mindnet can become:
A reusable “template” of neural health;
Broadcasted globally;
Entrained locally using other plasticity-friendly methods (like neurofeedback, sound therapy, or visual VR).

Summary: the loop
1. Hyperpolarized MRI captures therapeutic state;
2. AI compresses and distributes the signal;
3. Users receive it via sensory interfaces;
4. Hebbian learning rewires the brain to match;
5. The user learns to produce the healthy state independently over time.

This makes Hyperpolarized fMRI the “radio tower,” and Hebbian learning the receiver's internal engine—making MindNet a universal, adaptive, and scientifically grounded system.

### State of the Art

Previous patents and research efforts have explored brain-computer interfaces (BCIs), neurofeedback systems, and external stimulation (TMS, EEG) for neural interaction. However, these approaches: (i) require external stimulators to induce neural activity in the receiver; (ii) are prone to interference from external electronic devices and (iii) lack a natural neural decoding mechanism, making real-time information transfer difficult.

Existing BCI systems have explored EEG-based brain communication and fMRI-based neurofeedback, but these technologies are limited to basic motor commands or binary (yes/no) communication; do not allow continuous thought transmission in real-time and lack long-distance neural synchronization capabilities.

Some past studies indicated herewith have explored early forms of brain-to-brain communication, but they have been extremely limited.

In an University of Washington study of 2014 entitled “Direct Brain-to-Brain Communication with EEG and TMS”, researchers used EEG (electroencephalography) to extract signals and TMS (transcranial magnetic stimulation) to send basic motor commands. The limitations are: no real-time thought transmission, slow response time, required external stimulation.

The Harvard study “fMRI-Based Neural Synchronization Experiments” of 2019 has shown that fMRI detected brainwave synchronization between two people, but it was passive and not used for communication. Limitations: no AI optimization and no long-distance transmission.

MIT's study of 2021 “Non-Invasive BCI Systems for Basic Communication” used machine learning and EEG headsets to allow basic “yes/no” answers via brain signals, but it did not allow complex thought transmission.

No prior system has successfully combined AI, fMRI, hyperpolarized MRI (HP-13C MRI), and cloud-based neural transmission for brain-to-brain communication. Moreover, past research has been limited to EEG-based BCIs, small-scale fMRI studies, or external stimulation (TMS, tDCS), none of which achieve full long-distance, AI-optimized B2B communication.

The Alter Ego, a solution from MIT is now a start-up offering a device that “mind reads” to the public for the first time.

### SUMMARY OF THE INVENTION

The Mindnet Solution is a technology for real-time neural communication between two or more individuals using the integration of Pyneal Toolkit or proprietary software with Hyperpolarized MRI (HP-13C MRI) for neurodata transmission (DICOM) and natural neural synchronization decryption.

Unlike existing BCI approaches, this system does not require electrical, magnetic, or any other physical stimulation of the receiver's brain. Instead, it relies on natural neural resonance and shared cognitive representations to facilitate communication.

The invention can be used for medical applications, pain management, cognitive augmentation, direct thought-sharing, human communication and learning, entertainment, human-computer interaction, marketing and even sexual enhancement.

This invention presents a novel AI-enhanced brain-to-brain communication system, integrating:
Pyneal Toolkit or proprietary software for real-time fMRI signal acquisition;
hyperpolarized Carbon-13 MRI (HP-13C MRI) for metabolic tracking of cognitive effort;
AI-based Neural Synchronization Model to optimize brainwave alignment;
cloud-based encrypted neural data transmission for long-distance B2B communication using DICOM (Digital Imaging and Communications in Medicine), and
real-time neural plasticity training using reinforcement learning to improve efficiency over time.

Unlike prior approaches, this system does not require external stimulation (TMS, EEG, or electrical impulses) and instead relies on natural neural synchronization validated by metabolic activity.

### BRIEF DESCRIPTION OF THE DRAWINGS

For a better understanding and visualization of the object of the invention, it will now be described with reference to the appended figures, representing the technical effect obtained by means of an exemplary embodiment without limiting the scope of the present invention, wherein, schematically:

FIG. 1: presents a schematic illustration of the functioning of the object of the invention, using the hyperpolarized carbon-13 MRI; and

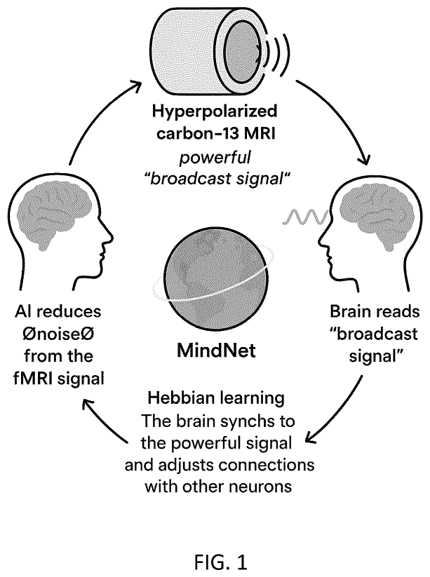

FIG. 2: indicates the functioning of the invention with the Hebbian learning system.

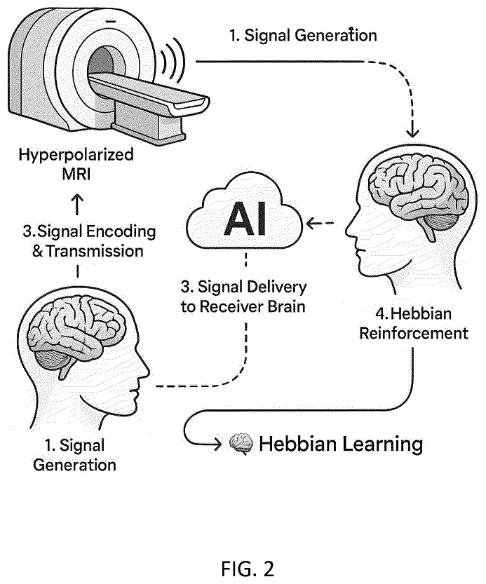

### DETAILED DESCRIPTION OF THE INVENTION

The Mindnet Solution is a system for direct neural interaction between individuals using real-time signal extraction, transmission, and neural synchronization; in other words, it's brain-to-brain communication technology.

The sender's brain Signal is broadcast through a functional magnetic resonance platform (MRI (fMRI)+HP-13C MRI) that scans the sender's neural and metabolic activity. The Pyneal toolkit or a proprietary software extracts real-time BOLD (blood-oxygen-level-dependent) and metabolic patterns from the sender's brain data, and an AI-based encoding software converts the neural signals into a format optimized for transmission.

The neural transmission & signal encoding device is a cloud-based neural network that securely transmits the encoded neural signals captured from the sender's brain to the receiver. The signal is encrypted using a brain-adaptive encoding method, ensuring that the receiver's nervous system can process and interpret.

Instead of using external devices to decode the signal, the receiver's brain naturally interprets the message based on shared neural representations. AI models assist in optimizing alignment and adjusting signal strength, improving natural interpretation.

### How the Receiver's Brain Decodes the Signal Naturally

Unlike traditional brain-computer interfaces (BCIs) that require external decoding hardware, this system leverages the nervous system's inherent ability to process and interpret neural patterns. Mirror neurons replicate and interpret brain activity from another person. The receiver's neurons fire in response to the sender's data, allowing perception of the transmitted information through patterns, repetition and synchronization. Example: The sender thinks about a moving object, and the receiver's visual cortex activates, as if seeing it themselves.

The receiver's brainwaves naturally align with the incoming signals, and a software analysis ensures real-time synchronization, improving accuracy.

The software tracks real-time metabolic activity in the receiver's brain; a real cognitive response confirms accurate interpretation of the incoming signal. This ensures that the receiver is truly experiencing the data of the sender, rather than random neural noise.

### AI-Powered Optimization of Neural Synchronization

To enhance natural interpretation, the system integrates AI-driven optimization models within the software. AI dynamically adjusts the transmission signal to match the receiver's cognitive rhythm. Predictive learning improves alignment over repeated transmissions. The system performs real-time feedback for signal clarity: the AI tracks metabolic activation in the receiver's brain, adjusting signal strength accordingly. Unsuccessful transmissions trigger adaptive AI corrections, improving future accuracy.

The receiver's brain activity is compared to the senders' in real time. High correlation scores indicate successful signal processing, and metabolic shifts in the receiver confirm true signal reception and interpretation.

### Applications & Use Cases

This system has applications in various fields, including:
Medical Communication for Non-Verbal Patients;
Enables locked-in syndrome and paralyzed individuals to communicate directly via brain signals;
Neurocognitive Enhancement & Augmented Collaboration;
Teams can share ideas without verbal language, enhancing problem-solving and creativity;
Dream Manipulation & Sleep-based Interaction;
Allows external influence over dream states, guiding dream experiences like mindfulness meditation;
Remote Brain-to-Brain Synchronization;
Long-distance communication without external devices or stimulation;
Sexual enhancement, and
Pain management.

The table below indicates how this technology differs from previous brain-computer interface (BCI) studies. While research has explored BCIs (Brain-Computer Interfaces) and basic brain-to-brain interfaces, there are key innovations that make this system unique.

| | | This New AI- |
|---|---|---|
| | Previous BCI/B2B | Enhanced B2B |
| Aspect | Research | Communication System |
| Technology | EEG, TMS, or basic | AI-enhanced fMRI + |
| Used | fMRI | HP-13C MRI + metabolic |
| | | tracking |
| Synchronization | Limited to basic EEG | AI-driven neural |
| Method | brainwave alignment | synchronization & |
| | | predictive adaptation |
| Transmission | Direct electrical/magnetic | Non-invasive, AI-optimized |
| Method | stimulation | fMRI transmission |
| Distance | Small-scale lab | Validated for long-distance |
| Feasibility | tests only | neural communication |
| Plasticity | Not considered in | AI-driven training for |
| Optimization | previous studies | long-term efficiency |
| Medical | Lacked metabolic | Uses metabolic imaging |
| Validation | tracking for real | (HP-13C MRI) to confirm |
| | responses | real neural activation |

This is the first technology to combine AI, metabolic imaging, and fMRI-based real-time brain-to-brain communication, making it a groundbreaking innovation. While previous research focused on direct electrical/magnetic stimulation of the brain for communication, the system described herein eliminates the need for external stimulation and uses AI-driven synchronization instead, which is not an obvious extension of current BCI technologies. Adding HP-13C fMRI for metabolic confirmation introduces a completely new method of validating true neural reception.

How Pyneal Toolkit Works with the B2B Communication Technology

The Pyneal Toolkit is an open-source Python-based real-time fMRI processing tool that plays a critical role in the AI-enhanced brain-to-brain (B2B) communication system. It enables real-time neural signal extraction, processing, and feedback, which is essential for synchronizing sender and receiver brain activity during B2B communication.

Pyneal is used in three key phases of the system:

1. Real-Time Neural Data Acquisition from the Sender: Pyneal connects to the HP-13C fMRI platform and extracts BOLD (blood-oxygen-level-dependent) signals and other information from the sender's brain. These signals represent real-time neural activity associated with what we could infer as data, or motor intentions. Pyneal processes this data instantly, converting it into a structured neural signal for transmission.

2. AI-Based Signal Processing & Transmission: Pyneal interfaces with AI models that refine, encode, and optimize the neural signal for transmission. The neural signal is encrypted and sent via a cloud-based transmission system to the receiver's brain. AI models ensure low-latency transmission, noise and high accuracy in neural synchronization.

3. Receiver's Brain Processing & Interpretation: Pyneal processes incoming neural signals from the sender using fMRI data from the receiver's brain. AI-powered synchronization models adjust signal encoding to ensure alignment with the receiver's brain activity. The receiver's brain naturally deciphers the incoming signal via mirror neurons and metabolic activity validation (HP-13C MRI).

Outcome: The receiver naturally interprets the sender's brain data without requiring external decryption devices.

| The Pyneal's specific | | |
|---|---|---|
| functions in | | |
| this technology | | |
| are shown | | |
| in the table | | Impact on Brain-to- |
| below.Function | Description | Brain Communication |
| Real-Time fMRI | Reads & processes | Enables real-time |
| Signal Extraction | signals from sender | neural communication |
| Neural Data | Filters noise & | Improves accuracy of |
| Preprocessing | optimizes signal clarity | transmitted data |
| AI Integration for | Uses AI to refine | Ensures high-quality |
| Signal Encoding | neural signals before | signal alignment |
| | transmission | |
| Low-Latency Data | Sends neural signals | Reduces delay in |
| Streaming | to cloud for processing | sender-receiver |
| | | synchronization |
| Receiver-Side | Analyzes incoming | Allows receiver's |
| Signal Decoding | neural signals | brain to naturally |
| | | interpret signals |
| Adaptive AI | Enhances sender/receiver | Improves long-term |
| Training | alignment over time | communication |
| | | efficiency |

How Pyneal Integrates with Other System Components

A. fMRI & HP-13C MRI: Pyneal extracts real-time neural activity from the plataform. HP-13C MRI provides metabolic tracking and other information to validate true cognitive engagement. Pyneal combines both datasets to ensure only authentic brain activity is transmitted.

B. AI Neural Synchronization Models: AI models analyze Pyneal-processed fMRI data and optimize neural synchronization scores. Reinforcement learning algorithms improve data transmission accuracy overtime. Pyneal continuously adapts the signal processing model based on AI feedback.

C. Secure Cloud-Based Transmission: Pyneal compresses and encrypts neural signals before sending them over a low-latency cloud network. The receiver's brain naturally decrypts the signal, ensuring privacy and security without external devices.

Outcome: Mindnet Solution enables seamless, real-time, AI-optimized brain-to-brain communication.

Mindnet advantages in B2B communication technology:

1) Real-Time Processing Capabilities: extracts real-time fMRI data from sender and receiver, processes neural signals instantly for seamless B2B communication, works in motion/different locations, and environmental conditions; 2) AI-Powered Optimization: automatically refines neural synchronization based on AI feedback, adapts signal encoding to maximize clarity and accuracy and continuously improves sender-receiver neural alignment over time; 3) Secure & Natural Interpretation: no external decryption devices needed, as the receiver's brain deciphers the signal naturally, HP-13C MRI confirms true cognitive engagement, preventing misinterpretation, and quantum-safe encryption ensures neural data privacy.

The Pyneal Toolkit plays a critical role in real-time fMRI processing and AI-driven neural synchronization for brain-to-brain communication (B2B). It acts as a real-time fMRI signal acquisition and processing engine that: extracts BOLD (blood-oxygen-level-dependent) signals from the sender's brain and other information; processes and encodes the neural signal using AI-based feature extraction; transmits the processed signal securely to the receiver's brain, and helps the receiver's brain naturally decrypt and interpret the message. As an outcome, Pyneal enables non-verbal, real-time, and long-distance thought transmission.

Below is described a step-by-step communication process using Pyneal.

A) Sender's Neural Signal Acquisition & Processing: functional MRI (fMRI) scans the sender's brain to detect real-time neural activity; hyperpolarized Carbon-13 MRI (HP-13C MRI) measures metabolic activity to confirm genuine thought engagement; Pyneal Toolkit captures the fMRI signal and applies real-time filtering to remove noise through DICOM; and AI models extract relevant neural features, ensuring only meaningful signals are transmitted. Result: Pyneal converts the sender's data into structured neural data for transmission.

B) Neural Signal Encoding & Secure Transmission: AI-based encoding optimizes neural signal format for low-latency transmission; Pyneal encrypts the neural data using a quantum-safe security protocol; the encoded signal is transmitted over a secure cloud-based system to the receiver. Result: neural data travels securely to the receiver's brain in milliseconds.

C) Receiver's Brain Processing & Interpretation: receiver's fMRI scans their brain activity to detect incoming neural signals; Pyneal compares the incoming signal with the receiver's baseline neural activity; mirror neurons in the receiver's brain activate, replicating the sender's brain data; HP-13C MRI verifies metabolic response, ensuring real neural synchronization; AI dynamically adjusts transmission parameters to optimize thought clarity; and the receiver's brain naturally deciphers the sender's brain data. Result: the sender's data is communicated to the receiver.

### Role of AI & Pyneal in Synchronizing Render-Receiver Brain Activity

To ensure accurate thought transmission, Pyneal works alongside AI-powered neural synchronization models: A) Real-Time Neural Feature Extraction: Pyneal extracts sender's BOLD signals in real time, and AI analyzes neural patterns to determine which signals encode meaningful data; B) Adaptive Transmission for Higher Accuracy: AI adjusts transmission speed and encoding based on receiver's brain state; Pyneal fine-tunes neural alignment using metabolic tracking feedback; C) Continuous Learning & Self-Optimization: AI trains on past synchronization patterns to improve accuracy over repeated sessions; and Pyneal integrates reinforcement learning to optimize sender-receiver neural alignment. Result: Over time, thought transmission becomes more accurate and subconscious.

### How Receiver and Sender are Synchronized in B2B Communication Using Pyneal

Synchronization between the sender and receiver in AI-enhanced brain-to-brain communication is achieved through a combination of functional MRI (fMRI), hyperpolarized MRI (HP-13C MRI), AI-driven neural synchronization models, and natural neural mechanisms such as mirror neuron activation and metabolic response matching.

To enable real-time brain-to-brain communication, the sender and receiver must achieve neural synchronization, which involves: capturing and processing neural signals from the sender in real time; encoding and transmitting these signals securely to the receiver; aligning the receiver's neural activity to match the sender's thought patterns; and validating synchronization through metabolic tracking to confirm true cognitive response. Outcome: the receiver naturally experiences and interprets the sender's data.

The synchronization occurs in three major phases:

A) Neural Signal Acquisition & Processing (Sender's Side): fMRI scans the sender's brain, capturing real-time neural activity; HP-13C MRI tracks metabolic responses to confirm the sender is actively engaged in data transmission; Pyneal extracts neural signals, applies preprocessing, and removes noise; and AI encodes the neural signal, optimizing it for synchronization with the receiver. Result: the sender's data is converted into a structured neural signal for transmission.

B) Signal Transmission & Synchronization Optimization: AI models analyze past sender-receiver interactions, adjusting the transmission format dynamically; neural data is encrypted and securely transmitted via a low-latency network; the receiver's fMRI scanner detects the incoming neural signal. Result: the signal reaches the receiver's brain with minimal latency, ensuring real-time thought synchronization.

C) Neural Alignment & Thought Interpretation (Receiver's Side): Receiver's fMRI detects neural activity changes in response to the incoming signal; AI compares receiver's brain activity with the sender's signal, adjusting in real time; mirror neurons in the receiver's brain activate, replicating the sender's thought patterns; HP-13C MRI confirms metabolic response alignment, ensuring the receiver is truly engaged in processing the thought; AI reinforces successful synchronization, continuously improving sender-receiver alignment; the receiver's brain naturally interprets the sender's data. Final outcome: communication achieved.

Key mechanisms that enable synchronization: A) AI-Based Neural Synchronization Modeling: AI learns sender-receiver neural patterns, adjusting transmission timing dynamically; reinforcement learning optimizes long-term sender-receiver alignment; B) Mirror Neuron Activation: mirror neurons replicate the sender's neural activity, allowing natural interpretation; C) Metabolic Response Matching (HP-13C MRI): HP-13C MRI tracks metabolic engagement in the receiver's brain, confirming true signal processing; D) Adaptive Feedback Loops: AI analyzes past interactions, fine-tuning sender-receiver synchronization overtime. Result: Data transmission improves with repeated sessions, leading to subconscious and automatic interpretation.

Summary of synchronization workflow: sender's actions receiver's response; neural signal acquisition fMRI captures data, AI encodes signals fMRI detects incoming signals; Transmission & Optimization Secure cloud transmission, AI adjusts encoding AI adapts receiver-side neural response; Neural Synchronization Mirror neurons activate, HP-13C MRI tracks metabolic signals receiver's brain deciphers and aligns with sender's thought; final Interpretation AI reinforces successful synchronization data is naturally acknowledged. Outcome: Real-time and accurate brain-to-brain communication.

### Using AI-Enhanced B2B Communication for Pain Management

Brain-to-brain (B2B) communication using Pyneal Toolkit, fMRI, HP-13C MRI, and AI-driven neural synchronization can be applied as an advanced pain management system by enabling neural modulation, shared perception of analgesic states, and real-time cognitive intervention.

How Brain-to-Brain communication can alleviate pain: the nervous system experiences and interprets pain through neural circuits in the brain, particularly in the: Somatosensory Cortex→Processes pain signals; Anterior Cingulate Cortex (ACC)→Governs pain perception and emotional suffering, and Periaqueductal Gray (PAG)→A key region for natural pain suppression (endogenous opioids). By synchronizing neural activity between a pain-free sender and a receiver experiencing pain, the system can: reduce pain perception by overriding nociceptive signals; activate neural pathways associated with analgesia in the receiver and modulate pain-related brain regions using shared neural states. Outcome: The receiver experiences a reduction in pain intensity and emotional distress.

Step-by-Step process of pain management via Brain-to-Brain Communication: A) Neural Signal Acquisition (Sender: Pain-Free State): sender is a trained individual (e.g., a meditator, athlete, or person with high pain tolerance); sender's brain data to capture low-pain or analgesic neural states; HP-13C MRI tracks metabolic activity in pain-processing regions to validate true analgesic response; AI extracts and encodes neural patterns from the sender's pain-free brain state. Result: Sender's pain-free state is converted into a structured neural signal. B) Neural Signal Transmission & Synchronization: AI optimizes the encoded signal for transmission to the receiver; the neural data is securely transmitted via a cloud-based network; receiver's detects the incoming neural signal and aligns brain activity accordingly. Result: The pain-free state is safely and accurately delivered to the receiver. C) Receiver's Brain Processing & Pain Reduction: AI compares the receiver's pain-related brain activity with the sender's analgesic state; mirror neurons and neural entrainment mechanisms activate, causing the receiver to mimic the sender's brain activity; HP-13C MRI confirms metabolic shift in pain-related regions, verifying real analgesic response; AI dynamically adjusts the transmission to optimize pain suppression. Final Outcome: The receiver experiences a reduction in pain and emotional distress.

The table below shows the mechanisms that enable pain suppression.

| | How it works in | |
|---|---|---|
| Mechanism | B2B Pain Management | Expected Effect |
| Neural Entrainment | Receiver's brainwaves | Pain perception is |
| | synchronize with | overridden |
| | the sender's | |
| | analgesic state | |
| Mirror Neuron | Receiver's neurons | Emotional suffering |
| Activation | mimic sender's pain- | from pain is |
| | free neural activity | reduced |
| Periaqueductal Gray | Shared neural states | Body produces |
| (PAG) Stimulation | trigger natural opioid | endorphins, reducing |
| | release | pain |
| Cognitive Reframing | AI adjusts neural | Pain is perceived as |
| via AI | alignment to suppress | less severe |
| | pain-related thought | |
| | patterns | |

Result: pain is naturally suppressed, reducing dependence on opioids or medications.

Advantages of AI-Driven B2B Pain Management: non-invasive—no need for implants or external pain relief devices; adaptive—AI learns and optimizes pain suppression over repeated sessions; drug-free—Reduces the need for opioids or pharmacological interventions, and customizable—Works for chronic pain, acute injuries, and post-surgical recovery. Real-World applications: Chronic Pain Therapy—helps patients with fibromyalgia, arthritis, and nerve pain; Post-Surgical Recovery—accelerates healing and reduces pain perception without opioids; Cancer Pain Management—Provides non-drug alternatives for pain relief; Military & Trauma Patients—Supports soldiers and accident victims in pain reduction. Outcome: AI-enhanced B2B communication revolutionizes pain management, reducing dependence on opioids while enhancing quality of life.

### Using AI-Enhanced B2B Communication for Sexual Experience Sharing

Brain-to-brain (B2B) communication using Pyneal Toolkit, fMRI, HP-13C MRI, and AI-driven neural synchronization can be applied to enhance, share, and synchronize sexual experiences between partners, even at a distance. This system allows one person to transmit sensory, emotional, and physical experiences directly to another person's brain, leading to intensified intimacy and shared pleasure without physical contact. The brain processes sexual arousal and pleasure through several key regions: Hypothalamus—controls arousal and hormone release; Insular Cortex—Processes physical sensations and pleasure; Amygdala—governs emotional connection and intimacy; Prefrontal Cortex—modulates sexual anticipation and imagination.

By synchronizing neural activity between partners, this system can: allow real-time sharing of pleasure and arousal; enhance emotional and sensory connection; create immersive intimate experiences, even at a distance; and simulate physical touch using AI-based neural stimulation. Outcome: Both partners experience pleasure as if they were physically together.

Step-by-step process of B2B sexual synchronization: A) Neural Signal Acquisition (Sender's Arousal State): sender's brain data to capture real-time sexual arousal signals; HP-13C MRI measures metabolic activity in sexual pleasure regions, confirming real engagement; Pyneal extracts and encodes the sender's arousal state into a structured neural signal; and AI filters the neural signal to enhance sensory clarity and remove noise. Result: The sender's pleasure state is transformed into a digital neural format. B) Signal Transmission & Neural Synchronization: AI optimizes the neural signal for real-time synchronization with the receiver; encrypted cloud transmission securely delivers the neural data; the receiver's fMRI detects the incoming neural pattern. Result: The sender's arousal reaches the receiver in milliseconds for real-time connection. C) Receiver's Brain Processing & Sexual Experience Sharing: AI compares the receiver's brain activity with the sender's arousal state; mirror neurons activate, replicating the sender's pleasure in the receiver's body; HP-13C MRI confirms metabolic alignment, ensuring true sensory synchronization; AI fine-tunes the transmission, adapting to real-time arousal changes; and the receiver experiences the sender's sensations as if they were physically together. Final outcome: the receiver feels the sender's touch, arousal, and emotional connection in real time.

The table below shows the mechanisms that enable neural synchronization of sexual pleasure.

| | How It Works in Sexual | |
|---|---|---|
| Mechanism | Experience Sharing | Expected Effect |
| Neural | Receiver's brainwaves | Sensory and emotional |
| Entrainment | synchronize with the | connection deepens |
| | sender's pleasure state | |
| Mirror Neuron | Receiver's neurons | Physical pleasure is |
| Activation | mimic the sender's | experienced remotely |
| | physical sensations | |
| Somatosensory | AI enhances real-time | Simulates physical |
| Cortex | neural encoding of | sensations |
| Stimulation | touch | |
| Emotional & Reward | Shared neural signals | Increased intimacy and |
| System Activation | stimulate dopamine and | bonding |
| | oxytocin release | |

Result: partners experience real-time intimacy and shared pleasure as if they were physically together.

Advantages of AI-Driven B2B sexual experience sharing: realistic & Immersive—simulates real touch, arousal, and emotional connection; Long-Distance Intimacy—enables partners to experience each other remotely; Customizable Stimulation—AI personalizes neural signals for tailored pleasure; Privacy-Secured—end-to-end encryption ensures private, secure experiences.

Real-World Applications: long-distance relationships—couples experience physical intimacy without geographic limits; disability & mobility assistance—helps individuals with mobility impairments experience intimacy; VR & neuro-intimate experiences—integrates with virtual reality for immersive neural intimacy; therapeutic & sensory rehabilitation—aids in restoring intimacy for trauma survivors. Outcome: AI-enhanced brain-to-brain intimacy revolutionizes human connection beyond physical limitations.

### Enhancing Learning Through AI-Enhanced B2B Communication Technology

Brain-to-brain (B2B) communication using Pyneal Toolkit, fMRI, HP-13C MRI, and AI-driven neural synchronization can be applied to accelerate learning, knowledge transfer, and skill acquisition by directly transmitting neural patterns from an expert (sender) to a learner (receiver).

How Brain-to-Brain Communication Enhances Learning: the human brain processes learning through neural circuits and memory networks, particularly in: Hippocampus—stores new knowledge and forms long-term memory; Prefrontal Cortex—responsible for reasoning, decision-making, and problem-solving; Motor Cortex & Cerebellum—essential for skill acquisition and procedural learning, and Mirror Neurons—allow learners to mimic expert thought patterns and motor actions. By synchronizing neural activity between an expert (teacher) and a learner (student), this system can: directly transmit knowledge and experiences to the learner; enhance skill acquisition through real-time brainwave synchronization; reduce cognitive load, allowing for faster understanding and retention; create immersive, experience-based learning without verbal or written instruction. Outcome: the learner absorbs complex knowledge and skills as if they personally experienced them.

Step-by-step process of brain-to-brain learning synchronization: A) Neural Signal Acquisition (Expert Knowledge Transmission): fMRI scans the expert's brain as they focus on a specific skill or concept; HP-13C MRI measures metabolic activity in knowledge-processing regions, ensuring deep engagement; Pyneal extracts and encodes the expert's neural patterns related to the subject matter; and AI refines the encoded signal for clarity and removes unnecessary noise. Result: The expert's neural state is transformed into a structured data format for direct knowledge transfer. B) Neural Transmission & Synchronization Optimization: AI optimizes the encoded neural signal for real-time synchronization with the learner; secure cloud transmission delivers the neural signal; the learner's fMRI detects and processes the incoming signal. Result: The learner's brain begins aligning with the expert's neural patterns. C) Learner's Brain Processing & Knowledge Absorption: AI compares the learner's neural activity with the expert's brain state; mirror neurons activate, replicating the expert's knowledge pathways; HP-13C MRI confirms metabolic engagement, ensuring real cognitive processing; AI dynamically fine-tunes the signal, enhancing the learner's neural absorption; and the learner experiences the knowledge as if they had learned it firsthand. Final Outcome: the learner internalizes the expert's knowledge and skills significantly faster than traditional learning.

The table below shows mechanisms that enable neural learning synchronization.

| | How It Works in | |
|---|---|---|
| Mechanism | B2B Learning | Expected Effect |
| Neural Entrainment | The learner's brain | Enhances deep |
| | synchronizes with the | learning |
| | expert's knowledge- | |
| | processing state | |
| Mirror Neuron | Learner's neurons mimic | Improves skill |
| Activation | expert thought processes | acquisition |
| Hippocampal Memory | AI optimizes memory | Boosts retention & |
| Encoding | consolidation | recall |
| Motor Cortex & | AI enhances neural | Speeds up physical |
| Procedural Learning | pathways for motor skills | skill learning |

Result: Learners gain knowledge and skills in real time, reducing years of study into hours.

Advantages of AI-Driven B2B Learning: faster Knowledge Acquisition—enables learners to absorb information rapidly; no Need for Traditional Study Methods—direct brain-to-brain knowledge transfer; Reduces Cognitive Effort—AI optimizes learning without overwhelming cognitive load, and Customizable Learning Paths—tailored to individual cognitive abilities and goals.

Real-World Applications: Medical Training—surgeons and doctors transfer procedural knowledge to trainees; STEM Education—direct brain-to-brain transmission of mathematics, physics, and engineering concepts; Language Learning—learners absorb new languages instantly by synchronizing with native speakers; Sports & Martial Arts Training—athletes learn new physical skills via neural synchronization; Music & Creativity Training—musicians absorb expert neural patterns for instrument mastery. Outcome: AI-enhanced B2B communication revolutionizes education by making learning faster, immersive, and subconscious.

### Enhancing Therapy & Mindfulness Through AI-Enhanced B2B Communication Technology

Brain-to-brain (B2B) communication using Pyneal Toolkit, fMRI, HP-13C MRI, and AI-driven neural synchronization can revolutionize therapy and mindfulness practices by allowing direct transmission of emotional states, cognitive patterns, and meditative states between individuals. This approach enables real-time emotional regulation, shared mindfulness experiences, and advanced neurofeedback therapy.

The brain regulates emotions, stress, and mindfulness states through key neural circuits: Prefrontal Cortex (PFC)—governs emotional control, mindfulness, and self-awareness; Amygdala—processes fear, anxiety, and emotional responses; Insular Cortex facilitates self-awareness and bodily sensations; Default Mode Network (DMN)—regulates inner thought patterns, essential for meditation.

By synchronizing brain activity between a therapist (or meditation expert) and a client, this system can: help clients experience calm and relaxation instantly; reduce anxiety and trauma symptoms through neural entrainment; train the brain for deeper mindfulness and emotional resilience; and provide immersive mental health therapy without verbal guidance. Outcome: The client's brain naturally enters a relaxed and emotionally balanced state.

Step-by-Step Process of Brain-to-Brain Therapy & Mindfulness Synchronization: A) Neural Signal Acquisition (Therapist or Meditation Expert): fMRI scans the therapist's or expert's brain while they experience a relaxed, mindful, or emotionally stable state; HP-13C MRI tracks metabolic activity in brain regions associated with mindfulness and emotional balance; Pyneal extracts and encodes the neural patterns associated with the expert's mindfulness or therapeutic state; and AI refines the neural data to ensure a clean, distraction-free signal for transmission. Result: The expert's mindful or calm brain state is digitally formatted for transmission. B) Neural Signal Transmission & Synchronization: AI optimizes the neural signal for synchronization with the client; secure cloud transmission delivers the encoded brain state to the client's fMRI system. Result: The client's brain starts aligning with the transmitted therapeutic neural state. C) Client's Brain Processing & Mindfulness Activation: AI compares the client's neural activity to the transmitted mindfulness state; mirror neurons activate, replicating the expert's brain activity; HP-13C MRI confirms metabolic alignment, ensuring the client truly engages in the process; AI fine-tunes the transmission for deeper emotional regulation; and the client experiences a mindfulness or relaxation state naturally. Final Outcome: The client enters a deep meditative state or experiences emotional relief without requiring traditional guidance.

The table below shows the key mechanisms that enable brain-to-brain therapy & mindfulness.

| | How it worksin | |
|---|---|---|
| | B2B Therapy & | |
| Mechanism | Mindfulness | Expected Effect |
| Neural Entrainment | The client's brain | Reduces stress & anxiety |
| | synchronizes with the | instantly |
| | therapist's relaxed | |
| | state | |
| Mirror Neuron | The client's neurons | Enhances meditative |
| Activation | mimic the expert's | awareness |
| | mindfulness state | |
| Prefrontal Cortex | AI optimizes thought | Improves cognitive control |
| Regulation | patterns for emotional | over emotions |
| | stability | |
| Insular Cortex | AI enhances self- | Deepens mindfulness |
| Stimulation | awareness and bodily | experience |
| | relaxation | |

Result: clients achieve deep relaxation, mental clarity, and emotional balance in minutes.

Advantages of AI-Driven B2B Therapy & Mindfulness: Instant Emotional Regulation—users experience calm and balance in real time; No Need for Verbal Guidance—mindfulness is transmitted directly to the brain; Enhances Trauma Therapy—supports PTSD and anxiety recovery without cognitive overload; Scalable & Accessible—works for remote therapy, stress relief, and emotional well-being.

Real-World Applications: PTSD & Trauma Therapy—helps patients relive calming experiences instead of traumatic ones; Meditation Training—enables instant deep meditative states without years of practice; Cognitive Behavioral Therapy (CBT) Enhancement—reinforces positive thinking patterns in real time; Emotional Balance & Mood Regulation—helps individuals overcome stress, depression, and anxiety. Outcome: AI-enhanced B2B therapy and mindfulness redefines emotional healing, reducing reliance on verbal therapy and medication.

### Enhancing Sensory Healing Through AI-Enhanced B2B Communication Technology

Brain-to-brain (B2B) communication using Pyneal Toolkit, fMRI, HP-13C MRI, and AI-driven neural synchronization can be used to accelerate sensory healing by transmitting healthy neural patterns from a donor (sender) to a patient (receiver). This allows individuals with sensory impairments, nerve damage, or trauma-related sensory disruptions to restore sensory functions by aligning their brain activity with that of a fully functioning neural system.

How B2B communication enhances sensory healing: the brain processes sensory information through specialized regions and neural pathways: Somatosensory Cortex—responsible for touch, pressure, pain, and proprioception (body awareness); Primary Visual Cortex—interprets visual signals from the eyes; Auditory Cortex—processes sounds and speech patterns; Olfactory Bulb & Gustatory Cortex—handle smell and taste perception; and Mirror Neurons—allow the brain to replicate and restore missing sensory functions when exposed to functional neural activity.

By synchronizing brain activity between a fully functional donor and a patient with sensory impairment, this system can: stimulate damaged or dormant sensory circuits; enhance neuroplasticity for sensory recovery; improve brain integration of prosthetic sensory devices (bionic limbs, cochlear implants, etc.); and support rehabilitation for stroke, nerve injuries, or sensory-processing disorders. Outcome: The receiver's brain learns to process sensory signals again, restoring lost or impaired sensations.

Step-by-Step Process of Sensory Healing Through Brain-to-Brain Communication: A) Neural Signal Acquisition from a Healthy Donor: fMRI scans the donor's brain while they experience a full sensory event (e.g., touch, sound, vision); HP-13C MRI measures metabolic activity to confirm deep sensory engagement; Pyneal extracts the donor's neural activity patterns related to sensory input; and AI refines the neural data, filtering out unnecessary noise. Result: The donor's healthy sensory experience is encoded into a structured neural signal for transmission. B) Neural Signal Transmission & Synchronization with the Patient: AI optimizes the neural signal for synchronization with the receiver's brain; encrypted cloud transmission securely delivers the neural signal; and the receiver's detects the incoming sensory patterns. Result: The receiver's brain begins aligning with the transmitted sensory experience. C) Patient's Brain Processing & Sensory Restoration: AI compares the patient's brain activity with the donor's sensory state; mirror neurons activate, replicating the donor's neural processing of sensory input; HP-13C MRI confirms metabolic engagement, ensuring real sensory integration; AI dynamically fine-tunes the transmission, optimizing sensory stimulation; and the patient begins to experience sensory feedback even if their sensory organ is impaired. Final Outcome: the patient regains sensory perception over repeated training sessions.

The table below shows the key mechanisms that enable sensory healing.

| | How it worksin B2B | |
|---|---|---|
| Mechanism | Sensory Healing | Expected Effect |
| Neural | The receiver's brain | Restores sensory |
| Entrainment | synchronizes with the | processing |
| | donor's sensory experience | |
| Mirror Neuron | The patient's neurons | Triggers sensory |
| Activation | mimic the donor's | pathways for |
| | sensory function | healing |
| Somatosensory | AI enhances real-time | Helps patients |
| Cortex | neural encoding of touch | feel again |
| Stimulation | and movement | |
| Neuroplasticity | AI optimizes neural | Enables long-term |
| Enhancement | pathways for adaptive | sensory healing |
| | sensory recovery | |

Result: patients restore or enhance sensory perception, even after nerve damage or sensory loss.

Advantages of AI-Driven B2B Sensory Healing: Restores Sensory Function Naturally—helps patients regain sensory experiences without invasive surgery; Personalized Therapy—AI customizes the sensory restoration process based on each patient's needs; Enhances Neuroplasticity—strengthens weakened or dormant neural pathways; and Non-Invasive & Drug-Free—no need for pharmaceutical interventions or risky procedures.

Real-World Applications: Restoring Touch After Nerve Damage—helps individuals with spinal cord injuries or peripheral nerve damage regain sensation; Enhancing Prosthetic Integration—improves bionic limb feedback by training the brain to feel artificial touch; Hearing Restoration for Cochlear Implant Users—enhances brain adaptation to electronic hearing aids; Rehabilitating Stroke Patients—supports stroke survivors in regaining lost sensory functions; and Treating Sensory Processing Disorders (Autism, PTSD, etc.)—helps individuals process sensory input more effectively. Outcome: AI-driven B2B sensory healing redefines rehabilitation, making sensory recovery faster and more effective.

### Optimizing Communication Through AI-Enhanced B2B Communication Technology

Brain-to-brain (B2B) communication using Pyneal Toolkit, fMRI, HP-13C MRI, and AI-driven neural synchronization can revolutionize human communication by enabling direct thought transmission, real-time neural synchronization, and enhanced cognitive alignment. This eliminates language barriers, misunderstandings, and processing delays, leading to faster, more accurate, and intuitive communication between individuals.

How B2B communication optimizes human interaction traditional communication methods rely on: Speech & Language—subject to misunderstanding, processing delays, and language barriers; Text-Based Messaging—slower, requiring cognitive effort to encode and decode messages; and Non-Verbal Cues—limited and sometimes ambiguous in conveying precise intent.

Brain-to-brain communication optimizes human interaction by: transmitting raw data without verbalization; eliminating the need for language translation; reducing communication latency to near-instantaneous speed; and improving cognitive synchronization between individuals for deeper understanding. Outcome: people can spread their brain data instantly, with high accuracy and without language constraints.

Step-by-Step Process of Optimized Communication Using B2B Technology: A) Neural Signal Extraction from the Sender: fMRI scans the sender's brain to capture real-time data and emotional intent; HP-13C MRI tracks metabolic activity to confirm active cognitive engagement; Pyneal extracts the sender's BOLD signals and other information, processing key data and emotional cues; and AI encodes the neural signal, optimizing it for clarity and relevance. Result: the sender's thought is converted into a structured neural signal for transmission. B) Thought Transmission & Neural Synchronization: AI refines and encrypts the neural data for seamless transmission; the neural signal is securely transmitted via cloud-based AI infrastructure; and the receiver's brain detects the incoming neural pattern. Result: the receiver's brain starts processing the sender's data before verbalization would normally occur. C) Receiver's Brain Processing & Thought Interpretation: AI compares the receiver's brain activity with the incoming signal and adjusts for accuracy; mirror neurons activate, replicating the sender's cognitive pattern; HP-13C MRI confirms metabolic alignment, ensuring genuine cognitive reception; AI dynamically fine-tunes the transmission, adapting to the receiver's neural state; and the receiver experiences the sender's thought directly, as if it were their own. Final Outcome: the receiver instantly understands the sender's thought without requiring speech, text, or gestures.

The table below shows the key mechanisms that optimize communication.

| | How it worksin B2B | |
|---|---|---|
| Mechanism | Communication | Expected Effect |
| Neural Entrainment | The receiver's brain | Improves mutual |
| | synchronizes with the | understanding |
| | sender's cognitive state | |
| Mirror Neuron | The receiver's neurons mimic | Reduces |
| Activation | the sender's thought | ambiguity and |
| | processes | misunderstanding |
| Language-Independent | AI optimizes neural patterns | Eliminates |
| Thought Encoding | for universal interpretation | language barriers |
| Real-Time Cognitive | AI enhances sender- receiver | Enables instant, |
| Alignment | neural synchronization | high-fidelity |
| | | thought sharing |

Result: communication becomes near-instantaneous, accurate, and universally understood.

Advantages of AI-Driven B2B Communication: Instant Thought Transmission—no need for speech or text, reducing cognitive load; Language-Free Communication—works universally without the need for translation; Emotionally Enhanced Interaction—captures emotional context for deeper connections; Reduced Misunderstanding—direct neural exchange ensures greater accuracy in intent; and Faster Decision-Making—enables teams to collaborate at the speed of thought.

Real-World Applications: Diplomatic & Cross-Cultural Communication—eliminates language barriers in global negotiations; Emergency & Military Operations—enables silent, secure thought transmission between teams; High-Stakes Business Decision-Making—enhances executive communication & rapid strategic alignment; Education & Skill Transfer—allows mentors to transmit knowledge instantly to students; and Enhanced Personal Relationships—enables deeper, non-verbal connection between partners. Outcome: brain-to-brain communication optimizes human interaction, making it faster, more efficient, and more intuitive than ever before.

### Enhancing Entertainment Through AI-Enhanced B2B Communication Technology

Brain-to-brain (B2B) communication using Pyneal Toolkit, fMRI, HP-13C MRI, and AI-driven neural synchronization will revolutionize the entertainment industry by enabling immersive, real-time shared experiences, direct emotional engagement, and cognitive synchronization between users and digital content.

How brain-to-brain communication transforms through traditional entertainment experiences (movies, music, games, VR) rely on external stimuli (visuals, sound, touch): with B2B communication, entertainment evolves into direct neural engagement, allowing: real-time emotional & sensory immersion in digital content; direct experience-sharing between users, removing the need for screens or controllers; fully interactive narratives where users “live” the story; and enhanced gaming experiences where players “think” actions instead of pressing buttons. Outcome: Users experience entertainment at a neurological level, making it hyper-realistic and deeply personal.

Step-by-Step process of brain-to-brain entertainment synchronization: A) Neural Signal Extraction from Digital Content: fMRI scans a content creator's brain as they experience emotions (joy, suspense, adrenaline); HP-13C MRI tracks metabolic activity to confirm deep engagement; Pyneal extracts the neural signals associated with the experience; and AI encodes the emotional and sensory patterns into a digital format. Result: The content creator's data and experiences are digitized for real-time transmission. B) Neural Synchronization & Real-Time Immersion: AI refines and encrypts the neural data for seamless integration with entertainment content; users connect via fMRI & HP-13C MRI, receiving real-time neural transmission; mirror neurons in the user's brain activate, mimicking the transmitted emotions; and HP-13C MRI confirms metabolic engagement, ensuring a real neural response. Result: the user feels the emotions and sensations of the content as if they were happening to them. C) Fully Immersive Experience & Interaction: AI adjusts neural stimulation dynamically, enhancing emotional depth; users experience content as direct data and feelings; neural synchronization allows real-time decision-making in interactive content; and multi-user shared experiences create direct brain-to-brain storytelling. Final Outcome: entertainment becomes a deeply personalized, shared, and emotionally immersive experience.

The table below shows the key mechanisms that optimize entertainment experiences.

| | How it works in B2B | |
|---|---|---|
| Mechanism | Entertainment | Expected Effect |
| Neural Entrainment | The user's brain | Immerses users fully into |
| | synchronizes with the | the story |
| | content's emotional & | |
| | sensory state | |
| Mirror Neuron | The user's neurons | Creates deep emotional |
| Activation | mimic the emotions of | connections |
| | characters | |
| Real-Time Cognitive | AI optimizes content | Enhances realism and |
| Synchronization | delivery for max | interaction |
| | immersion | |
| Multi-User Thought | Users experience a | Enables interactive |
| Sharing | collective brainwave | storytelling & shared |
| | network | gaming |

Result: entertainment moves beyond screens and controllers, evolving into fully immersive neural storytelling.

Advantages of AI-Driven B2B Entertainment: Emotionally Engaging—users directly, making movies & games more impactful; fully Interactive—no need for controllers, keyboards, or screens-users “think” actions; Hyper-Realistic Storytelling—AI ensures seamless immersion in narrative-driven experiences; Multi-User Neural Connection—enables shared data between participants; and Customizable Experience—AI personalizes emotional depth & intensity for each user.

Real-World Applications: Movies & Storytelling—viewers experience the characters' data directly; Gaming & eSports—players control actions with their minds & feel real-time in-game sensations; Virtual Reality & Augmented Reality—transforms VR into a full sensory experience without screens; Music & Live Concerts—listeners experience music emotionally and physically, synchronized with the artist's brain; Shared Dreamscapes—users co-create and experience dreams & virtual worlds together; and Books—users may read books together. Outcome: AI-driven B2B entertainment redefines engagement, making it more personal, immersive, and interactive than ever before.

Industries that Will be Affected by AI-Enhanced B2B Communication Technology

Brain-to-brain (B2B) communication using Pyneal Toolkit, fMRI, HP-13C MRI, and AI-driven neural synchronization will revolutionize multiple industries by eliminating communication barriers, accelerating knowledge transfer, and enabling direct thought exchange. Below is an analysis of key industries that will be impacted and how they will evolve with this technology.

Healthcare & Medicine—impact: telepathic communication between doctors and patients for better diagnosis. Direct brain-to-brain therapy for mental health (anxiety, PTSD, depression); pain management through neural synchronization with pain-free individuals; and accelerated recovery & neurorehabilitation for stroke and spinal cord injuries. Example use case: a paralyzed patient can regain sensations and mobility by synchronizing with a healthy donor's brain.

Education & Learning—impact: instant knowledge transfer between experts and learners; skill acquisition without traditional training methods (e.g., learning a language instantly); AI-optimized memory retention and cognitive enhancement; and faster workforce training and professional development. Example use case: a medical student instantly downloads a surgeon's experience into their brain before performing an operation.

Communication & Media—impact: instant, language-free communication across cultures; emotionally rich media experiences, where users “feel” stories instead of just watching; and thought-to-text and thought-to-video creation for faster content production. Example use case: international business meetings where all participants “think” in their native languages but understand each other instantly.

Entertainment & Gaming—impact: direct neural interface for gaming, eliminating controllers; emotional synchronization between users and characters in movies & VR.; and live concerts and music experienced as direct emotional transmissions. Example use case: a gamer experiences in-game movement and sensations as if physically present in the virtual world.

Business & Workforce Productivity—impact: faster decision-making through brain-to-brain corporate communication; collective problem-solving with shared neural intelligence; and instant data transfer between employees for task execution. Example: engineers working on different continents collaborate by synchronizing their data in real time.

Space Exploration & Deep—Space Communication—impact: instant brain-to-brain communication across vast distances; enhanced astronaut cognitive performance through neural training; and remote operation of spacecraft and robotic missions using thought control. Example use case: astronauts on Mars stay synchronized with Earth-based mission control using B2B thought-sharing.

Transportation & Smart Cities—impact: brain-controlled vehicles for seamless travel; traffic optimization through collective neural feedback; and neural authentication for accessing buildings, vehicles, and secure areas. Example use case: a driverless car anticipates and reacts to human intentions without requiring manual input.

### Creating B2B Communication Networks Using AI-Enhanced Neural Synchronization

Brain-to-Brain (B2B) networks using AI-driven neural synchronization, Pyneal Toolkit, fMRI, HP-13C MRI, and cloud-based transmission can establish real-time, large-scale cognitive sharing among multiple individuals or organizations. This would enable seamless knowledge transfer, collaborative problem-solving, and direct neural communication across multiple users.

What is a B2B network in the context of brain-to-brain communication?A Brain-to-Brain (B2B) Network is a system where multiple individuals: directly share data and cognitive states in real time. Collaborate without the need for verbal or text-based communication; synchronize problem-solving efforts through direct neural linkage; and enhance collective intelligence by pooling neural resources. Outcome: a fully connected neural internet, where individuals “upload” and “download” data instantly.

### Challenges & Solutions in B2B Neural Networks

The table below shows the main challenges and solutions in Brain-to-brain neural networks.

| Challenge | Solution |
|---|---|
| Neural Signal Clarity | AI filters noise & enhances signal |
| | processing |
| Privacy & Security | Quantum-safe brain encryption |
| | prevents unauthorized access |
| Data Overload | AI manages thought prioritization & |
| | filters unnecessary data |
| Latency Issues | Edge AI processing reduces |
| | transmission lag |
| User Adaptation | AI-driven training enhances neural |
| | synchronization over time |

Outcome: AI-driven B2B networks ensure seamless, efficient, and private brain-to-brain communication.

Future of B2B Neural Networks: Neural Internet—users upload & download knowledge instantly; AI-Neural Governance—collective decision-making via real-time thought consensus; Neural Blockchain Security—blockchain protects neural data integrity; and Decentralized Brain Networks—users connect to AI cloud servers without government control.

### CONCLUSION

A paradigm shift across industries, Mindnet, the first and only AI-enhanced brain-to-brain communication, will fundamentally transform how humans interact, work, and experience the world. It eliminates barriers of language, knowledge, and sensory perception, leading to faster, more efficient, and more immersive experiences across the world. Outcome: the future of communication shifts from words to pure thought exchange.

What is claimed is:

1. A system for direct neural interaction between at least one sender and at least one receiver using real-time signal extraction, transmission, and neural synchronization, the system comprising: a real-time Functional MRI (fMRI) processing via Pyneal Toolkit or proprietary software; a hyperpolarized Carbon 13 MRI (HP-13C MRI) module for tracking cognitive metabolic activity; an AI-powered Neural Processing Unit (NPU) to optimize neural transmission; a secure cloud-based neural data transmission network.

2. The system of claim 1, wherein the AI-powered Neural Processing Unit (NPU) synchs the sender in the Functional MRI (fMRI) and the hyperpolarized Carbon 13 MRI (HP-13C MRI) module to scan the at least one sender's neural and metabolic activity.

3. The system of claim 2, wherein after synchronization, the at least one receiver's brain processes the incoming signal without hardware external stimulators.

4. The system of claim 1, wherein the hyperpolarized Carbon 13 MRI (HP-13C MRI) is used to verify brain metabolic alignment between the at least one sender and at least one receiver and creates biological feedback for the AI-powered Neural Processing Unit (NPU) to adapt and optimize neural transmissions.

5. The system of claim 1, wherein the Pyneal toolkit or proprietary software extracts real-time BOLD (blood-oxygen-level-dependent) and metabolic patterns from the at least one sender, and an AI-based encoding system converts the neural signals into a format optimized for transmission to the at least one receiver.

6. The system of claim 1, wherein the AI-powered Neural Processing Unit (NPU) comprises a noise reduction function to isolate the therapeutic signature (e.g., low pain/low inflammation neural state) from irrelevant data (scanner artifacts, physiological rhythms, ambient EM, iatrogenic signals, weak microwave emissions).

7. The system of claim 1, wherein the AI-powered Neural Processing Unit (NPU) collaborates with the cloud-based neural network to transmit the neural signals to the at least one receiver.

8. The system of claim 7, wherein the neural signals are encrypted by the cloud-based neural network using a brain-adaptive encoding method.

9. The system of claim 1, wherein neural synchronization is optimized using reinforcement learning.

10. The system of claim 1, wherein brain-to-brain non-verbal, long-distance communication occurs without hardware external stimulation, via neural synchronization.
